(19) United States
(12) Patent Application Publication
(10) Pub. No.: US 2026/0117310 A1
(43) Pub. Date: Apr. 30, 2026

(54) METHYLATION SEQUENCING ASSAY TO ENABLE INTERPRETATION OF CLONAL HEMATOPOIESIS DYNAMICS

(71) Applicant: VANDERBILT UNIVERSITY, Nashville, TN (US)

(72) Inventors: Alyssa PARKER, Nashville, TN (US); Alexander BICK, Nashville, TN (US)

(21) Appl. No.: 19/370,917

(22) Filed: Oct. 28, 2025

Related U.S. Application Data
(60) Provisional application No. 63/713,250, filed on Oct. 29, 2024.

(51) Int. Cl. C12Q 1/6886; G16B 25/10

(52) U.S. Cl. CPC C12Q 1/6886; G16B 25/10; C12Q 2600/154

(57) ABSTRACT

In one aspect, the disclosure relates to methods for monitoring clonal hematopoiesis of indeterminate potential (CHIP) in a subject, the method including at least performing DNA methylation sequencing on DNA from the subject, performing cell-type deconvolution from the methylation data to estimate a first set of cell-type proportions in the subject, repeating the method to determine a second set of cell-type proportions in the subject, and monitoring a change in variant allele fraction (VAF) using methylation data collected during performance of the method in conjunction with the first set of cell-type proportions and the second set of cell-type proportions. Also disclosed are methods for assessing the performance of a drug for treating a blood cancer.

### CROSS REFERENCE TO RELATED APPLICATIONS

This application claims the benefit of U.S. Provisional Application No. 63/715,157 filed on Nov. 1, 2024, which is incorporated herein by reference in its entirety.

### STATEMENT REGARDING FEDERALLY SPONSORED RESEARCH OR DEVELOPMENT

This invention was made with government support under grant OD029586 awarded by the National Institutes of Health. The government has certain rights in the invention.

### BACKGROUND

Clonal hematopoiesis of indeterminate potential (CHIP) occurs when hematopoietic stem cells (HSCs) acquire a preleukemic driver mutation and produce blood cells that constitute a variant allele fraction (VAF) of ≥0.02 in peripheral blood. CHIP has a high prevalence (˜10% of people over the age of 60) and confers increased risk of hematologic malignancy, cardiovascular disease, and all-cause mortality. While some debate remains, most evidence suggests that the mechanism of disease risk occurs as a consequence of mutated HSCs preferentially producing pro-inflammatory myeloid cells. Current clinical practice relies on monitoring clone size through serial measurements of VAF using DNA sequencing. However, since the proportion of cells carrying CHIP mutations varies across cell types (FIG. 1A), fluctuations of cell-type proportions in response to other factors may confound clonal trajectory interpretation based solely on VAF (FIG. 1B).

Despite advances in CHIP diagnosis and monitoring research, there is still a scarcity of methods for calculating clone size via DNA sequencing that accounts for fluctuations in cell-type proportions. These needs and other needs are satisfied by the present disclosure.

### SUMMARY

In accordance with the purpose(s) of the present disclosure, as embodied and broadly described herein, the disclosure, in one aspect, relates to methods for monitoring clonal hematopoiesis of indeterminate potential (CHIP) in a subject, the method including at least performing DNA methylation sequencing on DNA from the subject, performing cell-type deconvolution from the methylation data to estimate a first set of cell-type proportions in the subject, repeating the method to determine a second set of cell-type proportions in the subject, and monitoring a change in variant allele fraction (VAF) using methylation data collected during performance of the method in conjunction with the first set of cell-type proportions and the second set of cell-type proportions. Also disclosed are methods for assessing the performance of a drug for treating a blood cancer.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1F show variation in cell-type proportions in blood samples exists in response to differentiation bias, exposures, and sampling methods. FIG. 1A) Schematic demonstrating hematopoietic cell differentiation bias in clonal hematopoiesis of indeterminate potential (CHIP). FIG. 1B) Variant allele fraction (VAF) and cell-type proportions vary over time in response to exposures. FIG. 1C) VAF over time for samples included in this study. Lines between points indicate that samples were taken from the same patient. FIG. 1D) Proportion of cells called as lymphocytes from complete blood counts (CBC) and from DNA methylation predictions. Circles indicate that samples were taken from patients with clonal hematopoiesis (CH), and triangles indicate that samples were taken from patients without CH. FIG. 1E) Proportion of cells called as granulocytes from CBCs and from DNA methylation predictions. FIG. 1F) Proportion of cells called as monocytes from CBCs and from DNA methylation predictions.

FIGS. 2A-2D show change in variation allele fraction (VAF) can be explained by fluctuations in cell-type proportions in short-term clonal hematopoiesis (CH). FIG. 2A) Methylation cell-type proportion predicted next VAF compared to measured next VAF for clones with mutations in TET2. FIG. 2B) Methylation cell-type proportion predicted next VAF compared to measured next VAF for clones with mutations in DNMT3A. FIG. 2C) Methylation cell-type proportion predicted next VAF compared to measured next VAF for clones with other CH mutations. FIG. 2D) Including cell-type proportions in a clone behavior schema reclassifies 57.1% of clones.

FIGS. 3A-3C show accuracy of next VAF predictions based on CBCs is lower than predictions based on methylation. FIG. 3A) CBC cell-type proportion predicted next VAF compared to measured next VAF for clones with mutations in TET2. FIG. 3B) CBC cell-type proportion predicted next VAF compared to measured next VAF for clones with mutations in DNMT3A. FIG. 3C) CBC cell-type proportion predicted next VAF compared to measured next VAF for clones with other CH mutations.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Aging is associated with the accumulation of somatic mutations across cells. Similar to other stem cells, hematopoietic stem cells (HSCs) accumulate mutations leading to increasing genetic diversity across an individual's lifetime. Individual HSCs are estimated to acquire 200 mutations per decade genome-wide, with 1 mutation per decade occurring within an exonic region. While the vast majority of such mutations do not have substantive impacts on cellular fitness, occasionally one such mutation may promote vitality and proliferation termed clonal hematopoiesis (CH).

CH has long been hypothesized as a key precursor in a sequential model of leukemogenesis. Age-related HSC clonal abnormalities in asymptomatic individuals was first recognized three decades ago through the analyses of non-random X-inactivation patterns derived from peripheral leukocytes of women. Population-based next-generation sequencing over the last decade has shown that CH is surprisingly common with approximately 1 in 10 asymptomatic adults older than 70 years affected. Using whole exome sequences of blood DNA originally aimed to discover rare germline disruptive coding alleles contributing to risk for common complex diseases, investigators employed methods to detect acquired mutations. ‘Clonal hematopoiesis of indeterminate potential’ (CHIP) is the presence of a hematologic malignancy driver mutation (typically in DNMT3A, TET2, ASXL1, JAK2) with high variant allele frequency in blood (i.e., >2%) indicative of clonality. While CHIP is a strong risk factor for hematologic malignancy, risk is not absolute with ˜0.5%/year progression from CHIP to hematologic malignancy.

A more surprising finding related to CHIP is that its implications for coronary artery disease may be a more important than hematologic malignancy. In several datasets, CHIP is associated with a 1.6-1.9-fold risk for coronary artery disease (CAD), and thus larger absolute risk increase for CAD compared to hematologic malignancy. Among asymptomatic individuals, individuals with CHIP have a greater burden of subclinical coronary atherosclerosis compared to those without. Consistent with the human observations, irradiated mice transplanted with Tet2−/− bone marrow versus transplanted with wild type bone marrow have a greater burden of supravalvular and descending aortic atherosclerosis. Both humans and mice with CHIP mutations in hematopoietic stem cells have greater concentrations of circulating inflammatory cytokines. Inhibition of the NLRP3 inflammasome mitigates atherosclerosis to a greater degree in irradiated mice transplanted with Tet2−/− bone marrow versus transplanted with wild type bone marrow. Similarly, genetic deficiency of IL6-receptor, in the NLRP3 pathway, through the presence of a common IL6R missense mutation in humans is associated with a greater reduction in cardiovascular disease risk among those with CHIP versus without. These data imply that for patients with CHIP, a tailored anti-inflammatory approach may be highly effective at addressing CHIP-associated cardiovascular disease risk. The increasingly robust therapeutic hypothesis is ripe for testing in placebo-controlled clinical trials.

Additional forms of CH have also been detected from the analysis of blood DNA. Larger chromosomal rearrangements, often term mosaic chromosomal alterations (mCAs) or clonal somatic copy number alterations, have been identified from large-scale blood DNA-derived genome-wide genotyping. While CHIP is strongly associated with myeloid malignancies, mCAs are strongly associated with lymphoid malignancies. Unlike CHIP, mCAs are not associated with CAD. Additionally, mCAs may represent more widespread immunologic dysfunction as they predict diverse incident cancers and infections.

Clonal hematopoiesis of indeterminate potential, or CHIP, is a common aging-related phenomenon in which hematopoietic stem cells (HSCs) or other early blood cell progenitors contribute to the formation of a genetically distinct subpopulation of blood cells. As the name suggests, this subpopulation in the blood is characterized by a shared unique mutation in the cells' DNA; it is thought that this subpopulation is “clonally” derived from a single founding cell and is therefore made of genetic “clones” of the founder.

Clonal hematopoiesis by itself is not considered to be a hematologic cancer; nevertheless, evidence is mounting that this condition may adversely affect human health. It has been proposed to label the group of individuals who have clonal hematopoiesis defined by a mutation in a malignancy-associated gene but without evidence of disease (such as cytopenia, dysplasia or immature “blast” cells in the bone marrow) as having CHIP. A clonal involvement (sometimes referred to simply as the size of a “clone”) of 2% of the blood has been tentatively proposed as a cutoff, though there is discussion that a lower floor that is more inclusive could also be appropriate. This cutoff may ultimately depend on whether clones must reach a certain size before influencing health. The level at which a clone begins to have a potential clinical impact is an open question, though there is already data to suggest larger clones have a larger effect on health.

The presence of clonal hematopoiesis/CHIP has been shown to increase blood cancer risk and is correlated with an increased risk of mortality overall. This is true both of clonal hematopoiesis with known candidate drivers as well as in cases without such drivers.

One area of health that CHIP has been definitively shown to influence is the risk of progression to blood cancer. In a given year, a tiny fraction of the general population will develop a hematologic cancer such as myelodysplastic syndrome (MDS) or AML; it is estimated that just 3 to 4 people per 100,000 will get MDS in a given year, and 4 people per 100,000 will develop AML. With CHIP, the risk of acquiring a hematologic malignancy like MDS or AML is increased more than 10-fold. Despite this increased risk, people with CHIP are still at low overall risk for developing a blood cancer, with only about 0.5-1.0% transformation per year.

A second area of health that may be affected by CHIP is the risk for heart attack and stroke. A strong association between CHIP and heart attack/ischemic stroke has been identified in multiple human genetic datasets, where CHIP was a stronger predictor of heart attack/stroke than if a patient 1) was a smoker, 2) had hypertension, 3) had high cholesterol, or 4) was overweight. In this study, which shows correlation but not causation, people with CHIP were 2.3 times more likely to suffer a heart attack, or 4.4 times as likely if the variant allele frequency in their blood was greater than 0.10, than matched controls without CHIP. It has also been found that there is an increased risk of cardiovascular mortality in patients who exhibit CHIP and receive self-derived stem cell transplantation. The idea of CHIP having a causal role in human heart attacks/strokes has been given support by a 2017 study that showed impairment of the Tet2 CHIP gene in mice causally led to accelerated atherosclerosis, and this finding in mice has been independently validated. The possibility of somatic mutations in the blood contributing not only to cancer risk but also to heart attack and stroke has generated much discussion in top-level scientific publications and a large multi-cohort study published in 2017 appears to confirm the causal link between CHIP and cardiovascular disease in humans.

In addition to its effects on those who would otherwise be considered healthy, CHIP may have implications in certain disease contexts. It has been shown that patients with CHIP who receive autologous stem cell transplantation (ASCT) as part of their treatment for lymphoma have worse outcomes than patients without CHIP. The poorer prognosis for these patients is due to both an increase in subsequent therapy-related myeloid neoplasms and increased risk for cardiovascular mortality.

Herein is disclosed a novel, cost-effective targeted enzymatic DNA methylation sequencing assay that captures 3.98 million CpGs at a cost of ˜$80 per sample. In some aspects, a subset of the 3.98 million CpGs can be tested such as, for example, about 3.75, 3.5, 3.25, 3, 2.75, 2.5, 2.25, 2, 1.75, 1.5, 1.25, or 1 million CpGs, or about 750,000, 500,000, 250,000, or 100,000 CpGs. This method offers a promising innovation for monitoring clonal trajectory.

In one aspect, disclosed herein is a method for monitoring clonal hematopoiesis of indeterminate potential (CHIP) in a subject, the method including at least the steps of:
(a) collecting a sample from the subject;
(b) extracting DNA from the sample;
(c) performing DNA methylation sequencing on the DNA to obtain methylation data;
(d) performing cell-type deconvolution from the methylation data to estimate a first set of cell-type proportions present in the subject;
(e) repeating steps (a)-(d) a second time to estimate a second set of cell-type proportions in the subject; and
(f) modeling change in variant allele fraction (VAF) using methylation data collected from steps (a)-(c), methylation data collected from step (e), the first set of cell-type proportions, and the second set of sell-type proportions.

In an aspect, the sample can be a cell-free sample or a whole blood sample. In another aspect, the sample can be another biological specimen that includes blood cells including, but not limited to, a peripheral blood mononuclear cell sample or a saliva sample.

In some embodiments, the DNA samples are obtained from one more cells in blood samples comprising hematopoietic stem cells (HSCs), committed myeloid progenitor cells having long term self-renewal capacity, or mature lymphoid cells having long term self-renewal capacity.

In some embodiments, the subject exhibits one or more risk factors of being a smoker, having a high level of total cholesterol or having high level of high-density lipoprotein (HDL).

In a further aspect, DNA methylation sequencing can be selected from whole genome bisulfite sequencing, reduced representation bisulfite sequencing, oxidative bisulfite sequencing, methylation sensitive restriction enzyme sequencing, direct methylation sequencing, enzymatic methyl-seq, or any combination thereof. In one aspect, the method further includes the step of enriching a plurality of methylated target DNA sequences prior to performing step (d). In one aspect, enriching can be accomplished by any means known in the art for the purpose of increasing an amount of signal from target DNA for easier readout and a lower error rate such as, for example, polymerase chain reaction amplification, In any of these aspects, cell-type proportions predicted in step (d) correlate with cell-type proportions determined by complete blood count (CBC) for at least one blood cell type such as, for example, lymphocytes, granulocytes, monocytes, or any combination thereof. In another aspect, subsets of these cell types can also be used in the disclosed method such as, for example, CD4+ T-cells, CD8+ T-cells, or the like. In any of these aspects, the method tests up to about 3.98 million different CpG methylation sites. In some aspects, a subset of the 3.98 million CpGs can be tested such as, for example, about 3.75, 3.5, 3.25, 3, 2.75, 2.5, 2.25, 2, 1.75, 1.5, 1.25, or 1 million CpGs, or about 750,000, 500,000, 250,000, or 100,000 CpGs.

In one aspect, in the disclosed method, linear modeling can be used in step (f) to identify relationship between change in VAF and change in cell-type proportion. In an alternative aspect, non-linear modeling, power modeling, exponential modeling, or any combination thereof can be used.

In an aspect, subject has a TET2 clone and an increase in monocytes can be used to predict a VAF obtained at a later time point, using an equation for linear modeling represented by

$${VAF}_{next} = {VAF}_{initial} ( 1.8 Δ M - 1.05 Δ L + 0.4 Δ G + 0.98 )$$

wherein VAFₙₑₓₜ is the VAF obtained at the later time point;
wherein VAFᵢₙᵢₜᵢₐₗ is a VAF obtained at an initial time point;
wherein ΔM is percent change in monocytes between the later time point and the initial time point;
wherein ΔL is percent change in lymphocytes between the later time point and the initial time point; and
wherein ΔG is percent change in granulocytes between the later time point and the initial time point.

In another aspect, the subject has a DNMT3A clone and a decrease in monocytes can be used to predict a VAF obtained at a later time point, using an equation for linear modeling represented by

$${VAF}_{next} = {VAF}_{initial} ( - 7 \text{.07} Δ M + 2 7.32 Δ L + 1 5.93 Δ G + 0.67 )$$

wherein variables are as described above.

In still another aspect, the subject has a clone with a mutation other than TET2 or DNMT3A such as, for example, at least one of ASXL1, CBL, GNAS, GNB1, IDH2, JAK2, KRAS, NRAS, PPM1D, SF3B1, TP53, or any combination thereof, and VAF change can be calculated using an equation for linear modeling represented by

$${VAF}_{next} = {VAF}_{initial} ( 12.47 Δ M + 4.54 Δ L + 2.91 Δ G + 0.88 )$$

wherein variables are as described above.

In some embodiments, the one or more mutations are frameshift mutations, nonsense mutations, missense mutations, or splice-site variant mutations.

In one aspect, the first set of cell-type proportions, the second-set of cell type proportions, or both, vary from a baseline in a healthy control subject due to co-occurrence at time of measurement of one or more inflammatory conditions or infections such as, for example, stomatitis, COVID infection, diverticulitis, sinus infection, or any combination thereof.

Also disclosed herein is method for evaluating performance of a drug for treating a blood cancer in a subject, the method including conducting the disclosed method to determine change in VAF in the subject,
wherein an initial VAF is determined before administering the drug,
wherein a later VAF is determined after administering the drug,
wherein the drug is considered successful if change in VAF is negative or no change is observed;
and wherein the drug is considered unsuccessful if change in VAF is positive.

Further in this aspect, the blood cancer can be chronic myelomonocytic leukemia, chronic myeloid leukemia, myelodysplastic syndrome, myelofibrosis, acute myeloid leukemia, or any combination thereof.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a mutation,” “a blood cell type,” or “a clone,” include, but are not limited to, mixtures, combinations, or populations of two or more such mutations, blood cell types, or clones, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “sample from a subject” refers to a tissue (e.g., tissue biopsy), organ, cell (including a cell maintained in culture), cell lysate (or lysate fraction), biomolecule derived from a cell or cellular material (e.g., a polypeptide or nucleic acid), or body fluid from a subject. Non-limiting examples of body fluids include blood, urine, plasma, serum, tears, lymph, bile, cerebrospinal fluid, interstitial fluid, aqueous or vitreous humor, colostrum, sputum, amniotic fluid, saliva, anal and vaginal secretions, perspiration, semen, transudate, exudate, and synovial fluid.

Unless otherwise specified, temperatures referred to herein are based on atmospheric pressure (i.e., one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Methods

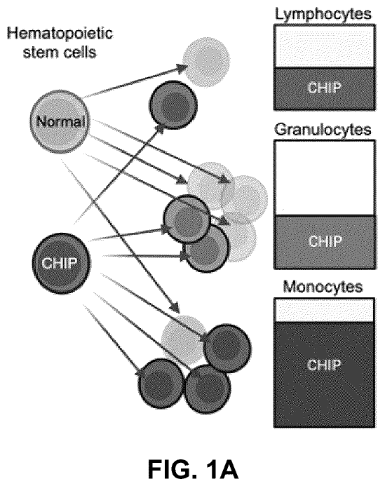

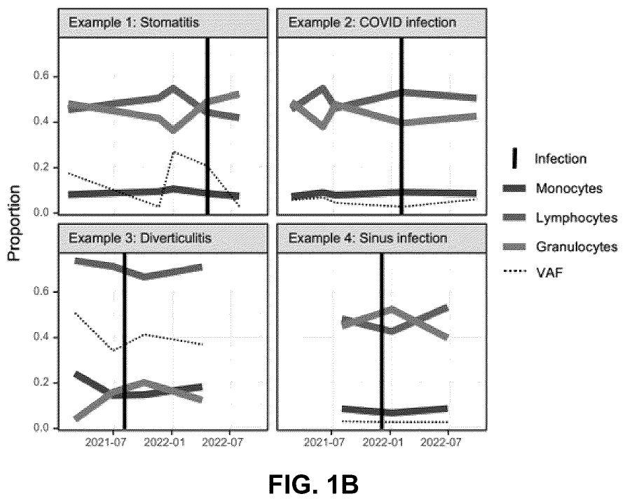

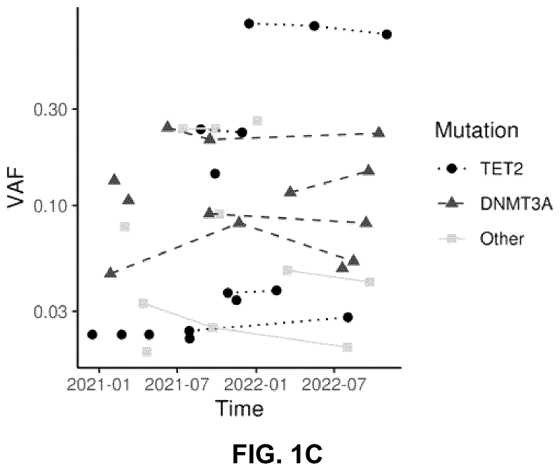

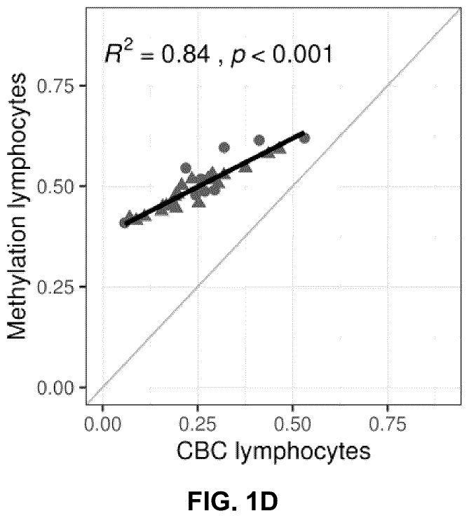

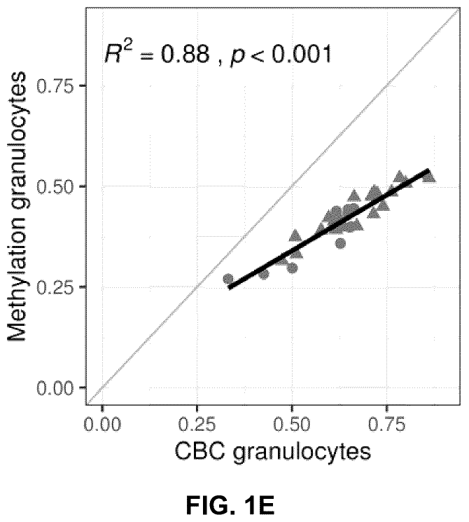

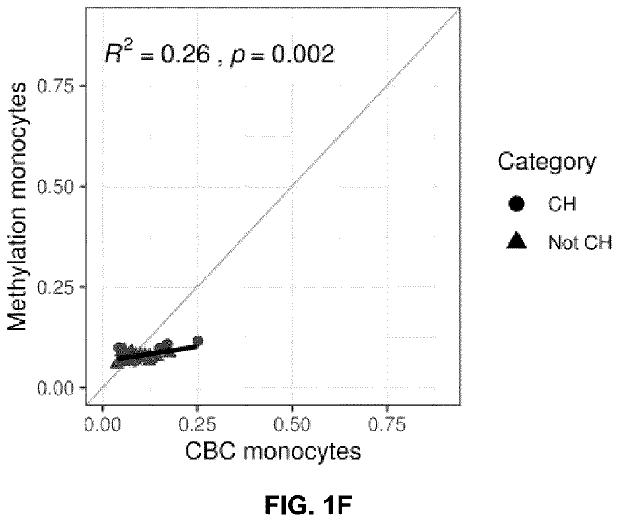

The study cohort consisted of 91 samples from 34 patients previously assessed for CHIP8. All patients in this study consented to study procedures through the Clonal Hematopoiesis and Inflammation in the Vasculature (CHIVE) cohort under Vanderbilt University Medical Center institutional review board approved research protocols (identifiers: 210022 and 201583). Blood samples were collected from participants between December 2020 and October 2022. Whole blood was isolated from K2 EDTA tubes and frozen at −20° C. DNA was extracted using standard protocols. It was observed that 24 patients carried a CHIP mutation with VAF 0.02 at at least one time point (FIG. 1C). Complete blood counts (CBCs) were obtained as part of routine clinical care.

DNA methylation sequencing was carried out using New England Biolabs Enzymatic Methyl-seq technology paired with a Twist Bioscience panel targeting 3.98 million CpGs. Sequencing was performed on an Illumina NovaSeq 6000 at 30×. Reads were aligned to the human reference genome (GRCh38), and methylation calls were made using Bismark. Cell-type deconvolution was performed with the R package methylCC.

Statistical analyses were conducted in R (version 4.3.2). Because mosaic loss of the Y chromosome can alter cell-type proportions significantly and becomes common amongst males with age, samples from male cases who were over the age of 74 at the time of blood draw were excluded from comparisons between CBC cell-type proportions and methylation predicted cell-type proportions.

Linear modeling was used to identify relationships between change in VAF and change in methylation-predicted cell-type proportions, stratified by driver gene. These linear relationships enabled the prediction of next VAF with high accuracy. Next VAFs were predicted with the following equations where ΔM represents percent change in monocytes, ΔL represents percent change in lymphocytes, and ΔG represents percent change in granulocytes: TET2 VAFₙₑₓₜ=VAFᵢₙᵢₜᵢₐₗ(1.8 ΔM−1.05 ΔL+0.40 ΔG+0.98); DNMT3A next VAFₙₑₓₜ=VAFᵢₙᵢₜᵢₐₗ(−7.07 ΔM+27.32 ΔL+15.93 ΔG+0.67); other mutations next VAFₙₑₓₜ=VAFᵢₙᵢₜᵢₐₗ(12.47 ΔM+4.54 ΔL+2.91 ΔG+0.88).

Clonal behavior was classified based on VAF only and based on a cell-type proportion informed metric. In the VAF-only strategy, clones with a percent change in VAF greater than 0.05 were classified as “expanding,” less than −0.05 as “shrinking,” and others as “stagnant.” In the cell-type proportion informed metric, the equations described above were used to predict percent change in VAF. Clones with a difference in predicted change VAF and measured change VAF greater than 0.05 were classified as “expanding,” less than −0.05 as “shrinking,” and all others as “stagnant.”

To benchmark performance of these methylation-based algorithms, the relationship between change in VAF and change in CBC-determined cell-type proportions was modeled using a cohort of 106 patients from Vanderbilt's biobank BioVU. Each patient had a CHIP clone at two timepoints. Next VAF was predicted from initial VAF and change in CBC-based cell-type proportions. In cases where next VAF was predicted to be higher than 1, next VAF was set to 1. Similarly, in cases where next VAF was predicted to be less than 0, next VAF was set to 0.

### Example 2: Results

The disclosed methylation-based cell-type proportion predictions correlated strongly with CBC results for lymphocytes (FIG. 1D; R²=0.84, p=2.65×10⁻¹⁴) and granulocytes (FIG. 1E; R²=0.88, p=4.31×10⁻¹⁶). Predictions for monocytes demonstrated a weaker, albeit still significant, correlation (FIG. 1F; R²=0.26, p=2.04×10⁻³).

Importantly, systematic differences were observed between methylation predictions and CBCs. These differences are likely attributable to cell loss during DNA extraction and library preparation steps. Specifically, it was found that granulocytes, which included neutrophils, basophils, eosinophils and mast cells, were consistently underrepresented in methylation-based predictions.

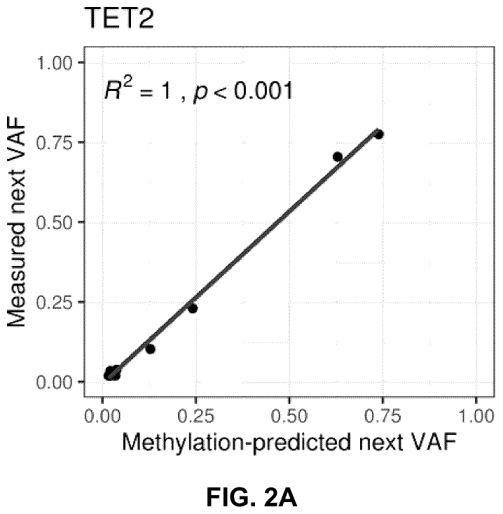

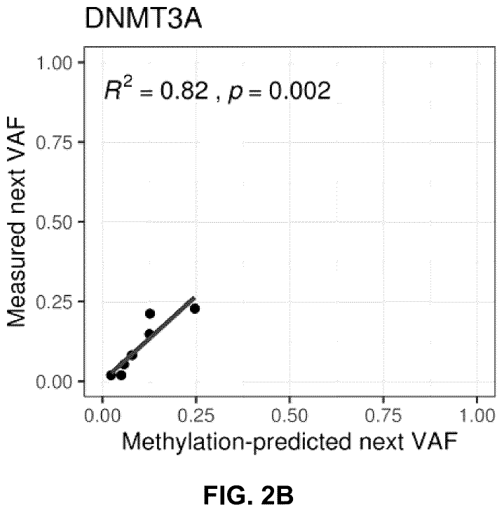

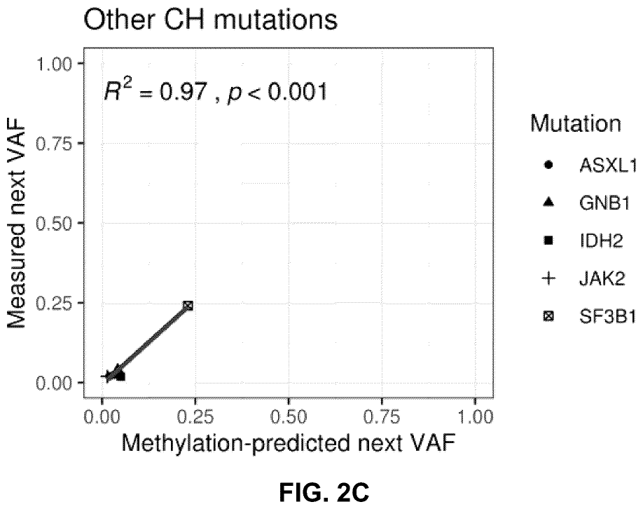

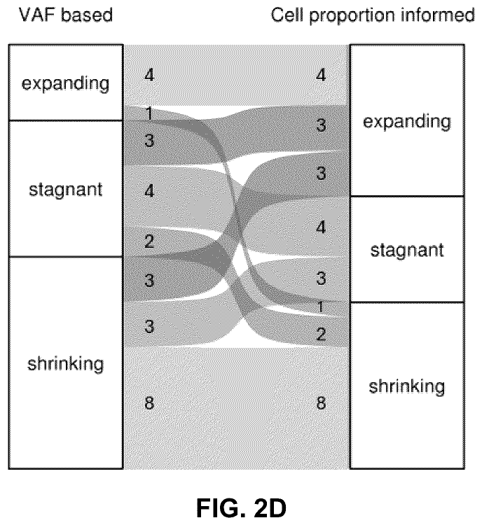

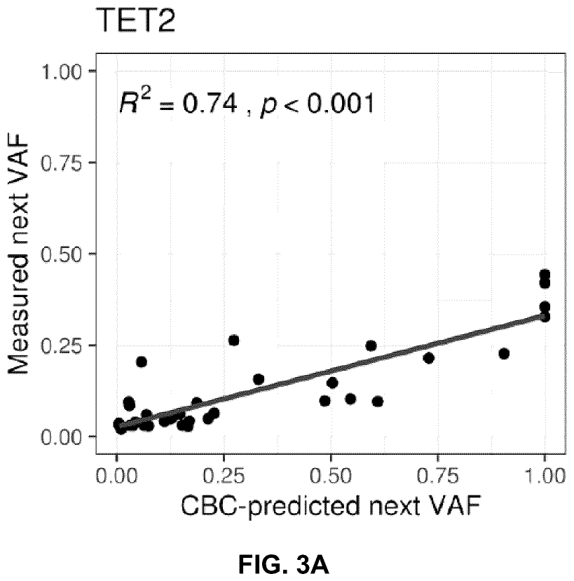

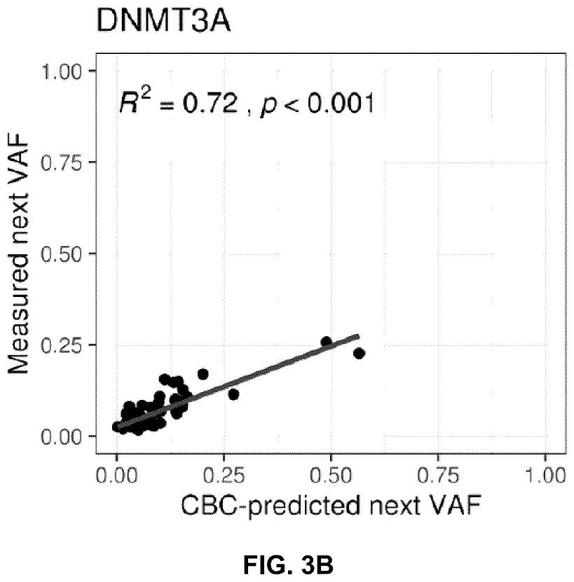

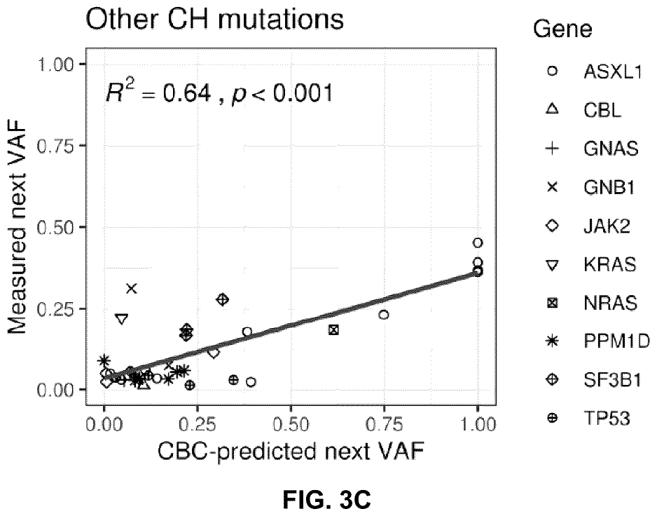

Linear relationships between change in VAF and change in cell-type proportions were identified that enabled the prediction of next VAF with high accuracy. The relationship for TET2 clones relied heavily on increases in monocytes (FIG. 2A; R²=0.996, p=7.42×10⁻¹⁶) Alternatively, the relationship for DNMT3A clones relied heavily on decreases in monocytes (FIG. 2B; R²=0.816, p=0.002). For clones driven by mutations in other genes, the cell groups were each important considerations (FIG. 2C; R²=0.974, p=3.88×10⁻⁵). Accuracy of these methylation-based predictions was much higher than CBC-based predictions (FIGS. 3A-3C; TET2 R²=0.74, p=3.25×10⁻¹¹; DNMT3A R²=0.72, p=3.55×10⁻¹⁷; Other R²=0.64, p=5.97×10⁻⁹).

It was found that fluctuations in cell-type proportions led to substantial reclassification of clonal trajectory. In 57.1% of paired samples (16/28 pairs), a cell-proportion-informed classification strategy resulted in a reclassification of clonal trajectory as compared to VAF-only classification (e.g., from “stagnant” to “expanding”) (FIG. 2D.

### Example 3: Discussion

These findings highlight limitations of relying solely on VAF measurements to monitor CHIP. Changes in VAF are insufficient to capture context, as CHIP biases differentiation trajectory of mutated cells. Misinterpretation of clonal trajectory in a clinical setting may lead to inaccurate assessments of risk.

Methylation sequencing offers several advantages over traditional monitoring approaches. First, cell-type proportions provide crucial context for interpreting VAF changes, which could help clinicians distinguish between clonal expansion due to altered bone marrow state and clonal expansion due to other factors. Equations were determined to contextualize clonal behavior, specific to driver genes. While high accuracy of these models was observed in this dataset, the degree of differentiation bias varies between clones and between individuals. Further work with variant-specific stratifications may enable greater generalizability.

Second, the methylation sequencing assay captures a more similar population of cells to DNA sequencing than CBC measurements. Systematic differences between cells captured in sequencing assays and CBCs were observed, supporting the idea that a portion of neutrophils is lost before sequencing. Furthermore, the disclosed models for predicting next VAF from methylation-predicted cell-type proportions performed much better than models based on CBC-determined cell-type proportions.

Third, the cost-effectiveness of this assay makes it feasible for implementation in clinical practice. This is particularly important given the increasing recognition of CHIP as a common age-related condition that requires long-term monitoring.

One limitation of this work is the low correlation of methylation-predicted monocyte proportions and CBC-determined monocyte proportions. In the original methylCC publication, the accuracy was evaluated for multiple methylation detection methods. The method most similar to NEB EMseq was Reduced Representation Bisulfite Sequencing (RRBS). For RRBS samples, the variability of methylCC predicted monocyte proportions was high, suggesting a limitation of methylCC.

In conclusion, the present study demonstrates that integrating methylation-based cell-type proportion data with VAF measurements can significantly enhance the interpretation of clonal trajectories. It should be emphasized that the above-described embodiments of the present disclosure are merely possible examples of implementations set forth for a clear understanding of the principles of the disclosure.

Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Bick, A. G. et al. Inherited causes of clonal haematopoiesis in 97,691 whole genomes. Nature 586, 763-768 (2020).
2. Bolton, K. L. et al. The Clinical Management of Clonal Hematopoiesis: Creation of a Clonal Hematopoiesis Clinic. Hematol. Oncol. Clin. North Am. 34, 357-367 (2020).
3. Buscarlet, M. et al. Lineage restriction analyses in CHIP indicate myeloid bias for TET2 and multipotent stem cell origin for DNMT3A. Blood 132, 277-280 (2018).
4. Cole, C. B. et al. Haploinsufficiency for DNA methyltransferase 3A predisposes hematopoietic cells to myeloid malignancies. J. Clin. Invest. 127, 3657-3674 (2017).
5. Connelly, A. N. et al. Optimization of methods for the accurate characterization of whole blood neutrophils. Sci. Rep. 12, 3667 (2022).
6. DeWitt, W. S. et al. Dynamics of the Cytotoxic T Cell Response to a Model of Acute Viral Infection. J. Virol. 89, 4517-4526 (2015).
7. Genovese, G. et al. Clonal Hematopoiesis and Blood-Cancer Risk Inferred from Blood DNA Sequence. N. Engl. J. Med. 371, 2477-2487 (2014).
8. Heimlich, J. B. et al. Multiomic Profiling of Human Clonal Hematopoiesis Reveals Genotype and Cell-Specific Inflammatory Pathway Activation. Blood Adv. 8, 3665-3678 (2024).
9. Heimlich, J. et al. Clonal Hematopoiesis and Inflammation in the Vasculature (CHIVE), a Prospective, Longitudinal Clonal Hematopoiesis Cohort and Biorepository. Blood 142, 5594 (2023).
10. Hicks, S. C. et al. methylCC: technology-independent estimation of cell type composition using differentially methylated regions. Genome Biol. 20, 261 (2019).
11. Izzo, F. et al. DNA methylation disruption reshapes the hematopoietic differentiation landscape. Nat. Genet. 52, 378-387 (2020).
12. Jaiswal, S. et al. Age-related clonal hematopoiesis associated with adverse outcomes. N. Engl. J. Med. 371, 2488-2498 (2014).
13. Jakubek, Y. A. et al. Genomic and phenotypic correlates of mosaic loss of chromosome Y in blood. Am J Hum Genet. 112, 276-290 (2025).
14. Ko, M. et al. Ten-Eleven-Translocation 2 (TET2) negatively regulates homeostasis and differentiation of hematopoietic stem cells in mice. Proc. Natl. Acad. Sci. U.S.A 108, 14566-14571 (2011).
15. Krueger, F. et al. Bismark: a flexible aligner and methylation caller for Bisulfite-Seq applications. Bioinformatics 27, 1571-1572 (2011).
16. Mack, T. et al. Cost-Effective and Scalable Clonal Hematopoiesis Assay Provides Insight into Clonal Dynamics. J. Mol. Diagn. 26, 563-573 (2024).
17. Moran-Crusio, K. et al. Tet2 Loss Leads to Increased Hematopoietic Stem Cell Self-Renewal and Myeloid Transformation. Cancer Cell 20, 11-24 (2011).
18. R Core Team. R: A Language and Environment for Statistical Computing. (R Foundation for Statistical Computing, Vienna, Austria, 2023).
19. Sehmi, R. et al. Allergen-induced increases in IL-5 receptor alpha-subunit expression on bone marrow-derived CD34+ cells from asthmatic subjects. A novel marker of progenitor cell commitment towards eosinophilic differentiation. J. Clin. Invest. 100, 2466-2475 (1997).
20. Serbina, N. V., et al. Selective Expansion of the Monocytic Lineage Directed by Bacterial Infection1. J. Immunol. 183, 1900-1910 (2009).
21. Smaaland, R., et al. Rhythms in human bone marrow and blood cells. Chronobiol. Int. 19, 101-127 (2002).
22. Steensma, D. P. et al. Clonal hematopoiesis of indeterminate potential and its distinction from myelodysplastic syndromes. Blood 126, 9-16 (2015).
23. Vaisvila, R. et al. Enzymatic methyl sequencing detects DNA methylation at single-base resolution from picograms of DNA. Genome Res. 31, 1280-1289 (2021).
24. Vlasschaert, C. et al. A practical approach to curate clonal hematopoiesis of indeterminate potential in human genetic data sets. Blood 141, 2214-2223 (2023).

What is claimed is:

1. A method for monitoring clonal hematopoiesis of indeterminate potential (CHIP) in a subject, the method comprising:
(a) collecting a sample from the subject;
(b) extracting DNA from the sample;
(c) performing DNA methylation sequencing on the DNA to obtain methylation data;
(d) performing cell-type deconvolution from the methylation data to estimate a first set of cell-type proportions present in the subject;
(e) repeating steps (a)-(d) a second time to estimate a second set of cell-type proportions in the subject; and
(f) modeling change in variant allele fraction (VAF) using methylation data collected from steps (a)-(c), methylation data collected from step (e), the first set of cell-type proportions, and the second set of cell-type proportions.

2. The method of claim 1, wherein the sample comprises a cell-free sample.

3. The method of claim 1, wherein the sample comprises a whole blood sample, a peripheral blood mononuclear cell sample, or a saliva sample.

4. The method of claim 1, wherein the DNA methylation sequencing comprises whole genome bisulfite sequencing, reduced representation bisulfite sequencing, oxidative bisulfite sequencing, methylation sensitive restriction enzyme sequencing, direct methylation sequencing, enzymatic methyl-seq, or any combination thereof.

5. The method of claim 1, further comprising enriching a plurality of methylated target DNA sequences prior to performing step (d).

6. The method of claim 1, wherein cell-type proportions predicted in step (d) correlate with cell-type proportions determined by complete blood count (CBC) for at least one blood cell type.

7. The method of claim 6, wherein the at least one blood cell type comprises lymphocytes, granulocytes, monocytes, or any combination thereof.

8. The method of claim 1, wherein the method tests up to about 3.98 million different CpG methylation sites.

9. The method of claim 1, comprising using linear modeling, non-linear modeling, power modeling, exponential modeling, or any combination thereof in step (f) to identify relationship between change in VAF and change in cell-type proportion.

10. The method of claim 9, wherein the subject has a TET2 clone and wherein an increase in monocytes can be used to predict a VAF obtained at a later time point.

11. The method of claim 10, wherein an equation for linear modeling is represented by
$${VAF}_{next} = {VAF}_{initial} ( 1.8 Δ M - 1.05 Δ L + 0.4 Δ G + 0.98 )$$
wherein VAFₙₑₓₜ is the VAF obtained at the later time point;
wherein VAFᵢₙᵢₜᵢₐₗ is a VAF obtained at an initial time point;
wherein ΔM is percent change in monocytes between the later time point and the initial time point;
wherein ΔL is percent change in lymphocytes between the later time point and the initial time point; and
wherein ΔG is percent change in granulocytes between the later time point and the initial time point.

12. The method of claim 9, wherein the subject has a DNMT3A clone and wherein a decrease in monocytes can be used to predict a VAF obtained at a later time point.

13. The method of claim 12, wherein an equation for linear modeling is represented by
$${VAF}_{next} = {VAF}_{initial} ( - 7 \text{.07} Δ M + 2 7.32 Δ L + 1 5.93 Δ G + 0.67 )$$
wherein VAFₙₑₓₜ is the VAF obtained at the later time point;
wherein VAFᵢₙᵢₜᵢₐₗ is a VAF obtained at an initial time point;
wherein ΔM is percent change in monocytes between the later time point and the initial time point;
wherein ΔL is percent change in lymphocytes between the later time point and the initial time point; and
wherein ΔG is percent change in granulocytes between the later time point and the initial time point.

14. The method of claim 9, wherein the subject has a clone with a mutation other than TET2 or DNMT3A.

15. The method of claim 14, wherein the mutation other than TET2 or DNMT3A comprises at least one of ASXL1, CBL, GNAS, GNB1, IDH2, JAK2, KRAS, NRAS, PPM1D, SF3B1, TP53, or any combination thereof.

16. The method of claim 15, wherein an equation for linear modeling is represented by
$${VAF}_{next} = {VAF}_{initial} ( 12.47 Δ M + 4.54 Δ L + 2.91 Δ G + 0.88 )$$
wherein VAFₙₑₓₜ is the VAF obtained at the later time point;
wherein VAFᵢₙᵢₜᵢₐₗ is a VAF obtained at an initial time point;
wherein ΔM is percent change in monocytes between the later time point and the initial time point;
wherein ΔL is percent change in lymphocytes between the later time point and the initial time point; and
wherein ΔG is percent change in granulocytes between the later time point and the initial time point.

17. The method of claim 1, wherein the first set of cell-type proportions, the second-set of cell type proportions, or both, vary from a baseline in a healthy control subject due to co-occurrence at time of measurement of one or more inflammatory conditions or infections.

18. The method of claim 17, wherein the one or more inflammatory conditions or infections comprises stomatitis, COVID infection, diverticulitis, sinus infection, or any combination thereof.

19. A method for evaluating performance of a drug for treating a blood cancer in a subject, the method comprising conducting the method of claim 1 to determine change in VAF in the subject,
wherein an initial VAF is determined before administering the drug,
wherein a later VAF is determined after administering the drug,
wherein the drug is considered successful if change in VAF is negative or no change is observed;
and wherein the drug is considered unsuccessful if change in VAF is positive.

20. The method of claim 19, wherein the blood cancer comprises chronic myelomonocytic leukemia, chronic myeloid leukemia, myelodysplastic syndrome, myelofibrosis, acute myeloid leukemia, or any combination thereof.
